(19) United States
(12) Patent Application Publication
(10) Pub. No.: US 2025/0000044 A1
(43) Pub. Date: Jan. 2, 2025

(54) A NOVEL QTL FOR PURPLE COLOR IN BRASSICA OLERACEA

(71) Applicant: Seminis Vegetable Seeds, Inc., St. Louis, MO (US)

(72) Inventors: Meinardus BOON, Scharwoude (NL); Sufei WANG, Santa Clara, CA (US)

(21) Appl. No.: 18/754,616

(22) Filed: Jun. 26, 2024

Related U.S. Application Data
(60) Provisional application No. 63/510,497, filed on Jun. 27, 2023.

(51) Int. Cl. A01H 6/20; A01H 5/12

(52) U.S. Cl. CPC A01H 6/203; A01H 5/12

(57) ABSTRACT

The present disclosure provides broccoli plants (Brassica oleracea var. italica) exhibiting a novel purple phenotype. Such plants may comprise novel introgressed genomic regions associated with the purple phenotype from Brassica oleracea var. capitata f. rubra. In certain aspects, compositions, including novel polymorphic markers and methods for producing, breeding, identifying, and selecting plants or germplasm with a purple phenotype are provided.

### CROSS-REFERENCE TO RELATED APPLICATIONS

This application claims priority to U.S. Provisional Patent Application No. 63/510,497, filed Jun. 27, 2023, the content of which is incorporated herein by reference in its entirety.

### INCORPORATION OF SEQUENCE LISTING

The sequence listing contained in the file named “BCS226311_US01.xml”, which is 7 kilobytes as measured in Microsoft Windows operating system and was created on May 28, 2024, is filed electronically herewith and incorporated herein by reference.

### FIELD

The present disclosure relates to the field of agriculture and, more specifically, to methods and compositions for producing broccoli plants with a unique purple phenotype. The purple phenotype is exhibited in broccoli plants and tissues including but not limited to the florets, main head, stems, beads, leaf petiole, and leaf midrib.

### BACKGROUND

One of the major goals in modern plant breeding programs is to improve existing plant varieties, especially vegetable crops. In addition to identifying alleles that confer a desirable phenotype, specific markers linked to such alleles facilitate their introduction into cultivated lines. Marker-assisted selection (MAS) in plant breeding methods has made it possible to select plants based on genetic markers linked to traits of interest, in this case, a purple phenotype. However, identification of markers for tracking and/or introducing desirable traits in plants requires significant effort and as such, the markers are often unavailable even if the gene associated with the trait has been characterized. The difficulty in identifying markers is also complicated by factors such as polygenic or quantitative inheritance, epistasis and an often incomplete understanding of the genetic background underlying expression of a desired phenotype.

### SUMMARY

In one aspect, the present disclosure provides an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele. In some embodiments, said recombinant chromosomal segment comprises a marker selected from the group consisting of marker M1 (SEQ ID NO:1), marker M2 (SEQ ID NO:2), marker M3 (SEQ ID NO:3), and marker M4 (SEQ ID NO:4) on chromosome 6. In some embodiments, said plant is homozygous for said recombinant chromosomal segment. In other embodiments, said plant is heterozygous for said recombinant chromosomal segment. In further embodiments, a sample of seed comprising said recombinant chromosomal segment has been deposited under NCMA Accession No. 202211063.

In addition, the present disclosure provides a plant part of an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele. In some embodiments, said plant part is a cell, a seed, a root, a stem, a stalk, a leaf, a bud, a flower, a floret, a head, an ovule, or pollen. The present disclosure also provides a seed that produces the broccoli plants described herein.

The present disclosure provides a recombinant DNA segment (which may be referred to as a nucleic acid molecule) comprising an allele from Brassica oleracea var. capitata f. rubra, wherein said allele confers to a broccoli plant a purple phenotype when present in said plant. In some embodiments, said recombinant DNA segment comprises a sequence selected from the group consisting of SEQ ID NOs: 1, 2, 3, and 4. In other embodiments, said recombinant DNA segment is further defined as comprised within a plant, plant part, plant cell, or seed. In further embodiments, a sample of seed comprising said recombinant DNA segment has been deposited under NCMA Accession No. 202211063.

In yet another aspect, the present disclosure provides a nucleic acid molecule comprising an allele conferring to a broccoli plant a purple phenotype, wherein the nucleic acid molecule is obtainable or can be obtained from seed deposited under NCMA Accession No. 202211063, and wherein the nucleic acid molecule comprises at least one marker selected from the group consisting of marker M1 (SEQ ID NO:1), marker M2 (SEQ ID NO:2), marker M3 (SEQ ID NO:3), and marker M4 (SEQ ID NO:4). In further embodiments, SEQ ID NO: 1 comprises a C at position 61; SEQ ID NO:2 comprises a C at position 61; SEQ ID NO:3 comprises a T at position 61, and SEQ ID NO: 4 comprises a G at position 61.

In addition, the present disclosure provides a cell of an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele. The present disclosure also provides tissue culture comprising a cell of an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele.

The present disclosure also provides a method for producing an elite broccoli plant exhibiting a purple phenotype, comprising introgressing into said plant an allele from Brassica oleracea var. capitata f. rubra within a recombinant chromosomal segment flanked in the genome of said plant by marker M1 (SEQ ID NO:1) and marker M4 (SEQ ID NO:4) on chromosome 6, wherein said allele confers to said plant a purple phenotype compared to a plant not comprising said allele, and wherein said introgressing comprises marker-assisted selection. In some embodiments, said introgressing comprises: a) crossing a plant comprising said chromosomal segment with itself (also referred to as selfing) or with a broccoli plant of a different genotype to produce one or more progeny plants; and b) selecting a progeny plant comprising said chromosomal segment. In other embodiments, selecting a progeny plant comprises detecting nucleic acids comprising marker locus M1 (SEQ ID NO:1), marker locus M2 (SEQ ID NO:2), marker locus M3 (SEQ ID NO:3), or marker locus M4 (SEQ ID NO:4). In other embodiments, the progeny plant is an F2-F6 progeny plant. In further embodiments, said crossing comprises backcrossing. The present disclosure further provides broccoli plants obtainable by the methods provided herein.

The present disclosure further provides a method of producing food or feed comprising obtaining an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele, or a part thereof, and producing said food or feed from said plant or part thereof.

The present disclosure provides a method of selecting a broccoli plant exhibiting a purple phenotype, comprising: a) crossing an elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele with itself (also referred to as selfing) or with a second broccoli plant of a different genotype to produce one or more progeny plants; and b) selecting a progeny plant comprising said recombinant chromosomal segment. In some embodiments, selecting said progeny plant comprises detecting a marker locus genetically linked to said recombinant chromosomal segment. In other embodiments, selecting said progeny plant comprises detecting a marker locus within or genetically linked to a chromosomal segment flanked in the genome of said plant by marker locus M1 (SEQ ID NO:1) and marker locus M4 (SEQ ID NO: 4) on chromosome 6. In some embodiments, selecting a progeny comprises detecting nucleic acids comprising marker locus M1 (SEQ ID NO:1), marker locus M2 (SEQ ID NO:2), marker locus M3 (SEQ ID NO:3), or marker locus M4 (SEQ ID NO:4). In further embodiments, said progeny plant is an F2-F6 progeny plant. In further embodiments, producing said progeny plant comprises backcrossing. The present disclosure further provides broccoli plants obtainable by the methods provided herein.

In another aspect, the present disclosure provides a method of selecting a broccoli plant or broccoli seed, comprising a) detecting in a population of broccoli plants or broccoli seeds a broccoli plant or broccoli seed comprising an allele from Brassica oleracea var. capitata f. rubra that confers to the plant a purple phenotype compared to a plant not comprising the allele and b) selecting the broccoli plant or broccoli seed comprising the allele from Brassica oleracea var. capitata f. rubra that confers to the plant a purple phenotype. In some embodiments, the allele is located on chromosome 6. In further embodiments, the allele is linked to at least one of markers: M1 (SEQ ID NO:1), M2 (SEQ ID NO:2), M3 (SEQ ID NO:3), and M4 (SEQ ID NO:4) on chromosome 6. In some embodiments, the broccoli plant is homozygous for the allele from Brassica oleracea var. capitata f. rubra. In other embodiments, the broccoli plant is heterozygous for the allele from Brassica oleracea var. capitata f. rubra. In further embodiments, M1 comprises a C at position 61 of SEQ ID NO:1; M2 comprises a C at position 61 of SEQ ID NO:2; M3 comprises a T at position 61 of SEQ ID NO:3; and M4 comprises a G at position 61 of SEQ ID NO: 4. In yet further embodiments, the broccoli plant or broccoli seed is an elite plant or a seed of an elite plant. In yet another aspect, the present disclosure provides a plant identifiable by the above-referenced method of selecting.

### BRIEF DESCRIPTION OF THE DRAWINGS

FIG. 1: Shows images of broccoli plants comprising the novel purple phenotype locus on chromosome 6 from Brassica oleracea var. capitata f. rubra (left side) and commercially available purple sprouting type broccoli (right side). The dark portions in the images represent purple coloration whereas the lighter portions represent green coloration.

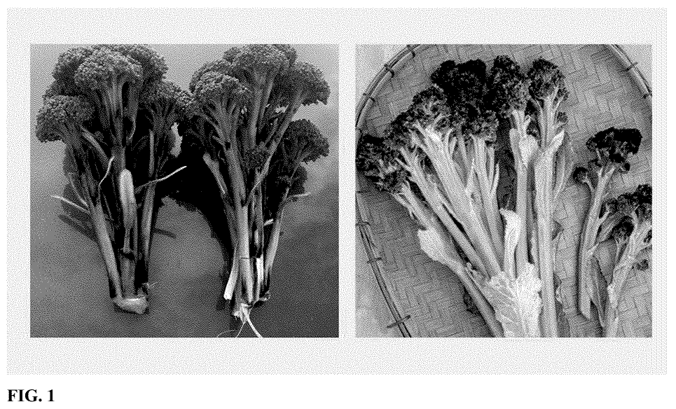

### DETAILED DESCRIPTION

To meet the dietary needs of a growing world population, agricultural breeding programs have primarily focused on accelerating and increasing crop production. While development of strategies to maximize crop yields is still a major objective in agricultural research, many breeders are now shifting their objective towards the development of crop varieties of high quality. Increased public awareness of the advantages of diets rich in richly colored vegetables has resulted in more consumers choosing produce based on its color and perceived health benefits.

Broccoli (Brassica oleracea var. italica) is one of the most popular vegetables among health-conscious consumers as a source of high levels of vitamins, antioxidants, and anticarcinogenic compounds. Although all parts of a broccoli plant are edible, the plant is typically harvested for young inflorescences. Broccoli is generally classified as either a “heading” type or a “sprouting” type, depending on the degree of branching exhibited and the size of the inflorescences. The heading broccoli type is usually green in color and distinguished by a single large terminal inflorescence whereas the sprouting type is characterized by many smaller lateral inflorescences that may be green, white, or purple in color. It is a cool weather crop that is typically grown in the spring or fall seasons.

Purple broccoli varieties have increased in popularity due to consumers' perception of the color being not only visually attractive but also associated with health benefits. In fruits and vegetables, plant tissues that are purple, blue, or red in color signifies the presence of high levels of anthocyanin in the tissue. Anthocyanins belong to a class of flavonoid compounds that not only impart color to plants, but also play an important role in protection of plants against a variety of biotic and abiotic stresses. For humans, anthocyanins are associated with antidiabetic, anticancer, anti-inflammatory, antimicrobial, and anti-obesity effects, as well as prevention of cardiovascular diseases. For these reasons, an important target in crop breeding is increasing the anthocyanin content in plants.

Purple broccoli has typically been limited to the sprouting type, where the plant is entirely green with the exception of the purple-colored heads. Recently, purple broccolini has been developed and is distinct from both the heading and sprouting broccoli types. This variety, disclosed in WO2018085646 A1, is a hybrid of standard broccoli (Brassica oleracea var. italica) crossed with Chinese kale (Brassica oleracea var. alboglabra, also known as Chinese broccoli, gai-lan, and kai-lan) and forms small purple florets with long, slender stalks that have some purple coloration. There also exists a variety of broccoli that is visually very similar to traditional green heading broccoli (i.e. having a domed head and fine grains) but has purple grains and some purple coloration in the stem. In these publicly known purple broccoli varieties, there is a high degree of variability in the intensity of the purple color and its expression throughout the plant tissues. There exists a need for broccoli plants that fully purple in color, wherein the color is intense and widespread over the majority of the plant.

The present inventors surprisingly discovered a novel purple phenotype locus on chromosome 6 from red cabbage (Brassica oleracea var. capitata f. rubra) which confers an unexpectedly intense and consistent purple color to the florets, flower beads, stem, main head, leaf petiole, and leaf midrib of broccoli plants comprising said locus. The phenotype conferred by this locus enables its use in dual-purpose broccoli varieties. Plants of such varieties produce a deeply branched head with a cleaner stem that can be harvested in its entirety like a floretting type and produce side shoots that can be harvested as stem broccoli. The purple color conferred by the purple phenotype locus is shown, for example, in the Royal Horticultural Society color chart, 6th edition, in color groups IX-Violet and X-Purple. The color can be selected from the group comprising 83A, 86A, N92A, N92B, N92C, N92D, N77A, 79A, 79B, 79C, N79A, N79B, N79C, N79D, or N81A, or can be selected from the group comprising 83A, 86A, N92A, 79A, N79A, N79B, or N79C. The purple phenotype is present when a broccoli plant is either heterozygous or homozygous for the introgression, but the phenotype is more intense when the locus is homozygous.

### I. Genomic Regions, Alleles, and Polymorphisms Associated with a Purple Phenotype in Broccoli Plants

One of skill in the art will understand that interval values may vary based on factors such as the reference map that is used, the sequencing coverage and the assembly software settings. However, such parameters and mapping protocols are known in the art and one of skill in the art can use the marker sequences provided herein to physically and genetically anchor the introgressions described herein to any given map using such methodology. The novel introgressions of the present disclosure confer a unique purple phenotype that is characterized by the whole plant exhibiting a purple color that is richer and more widespread than the previously disclosed purple broccoli varieties.

The Taqman markers provided herein can be used individually or in various combinations to identify, track, or introgress the locus that confers the purple phenotype. The purple phenotype QTL is defined by boundary markers M1, a [C/T] SNP change at position 18,177,971 bp of the public Brassica oleracea reference genome, and M4, a [G/T] SNP change at position 41,865,934 bp of the public Brassica oleracea reference genome. Interstitial markers M2, a [C/T] SNP change at position 29,582,267 bp of the public Brassica oleracea reference genome, and M3, a [T/G] SNP change at position 39,222,337 bp of the public Brassica oleracea reference genome, can be used to identify, track, or introgress the purple phenotype locus on chromosome 6. The public genome positions are based on version 1.0 of the Brassica oleracea cultivar HDEM reference genome found online at the Brassicaceae Database (brassicadb.cn).

### II. Introgression of Genomic Regions Associated with a Purple Phenotype in Broccoli

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

The present disclosure provides novel accurate markers for identifying and tracking introgression of one or more of the genomic regions disclosed herein from a Brassica oleracea L. var. capitata f. rubra plant into a cultivated broccoli line. The present disclosure further provides markers for identifying and tracking the novel introgressions disclosed herein during plant breeding, including the markers set forth in Table 1.

Markers within or linked to any of the genomic intervals of the present disclosure can be used in a variety of breeding efforts that include introgression of genomic regions associated with a purple phenotype into a desired genetic background. For example, a marker within 20 cM, 15 cM, 10 cM, 5 CM, 2 cM, or 1 cM of a marker associated with the purple phenotype described herein can be used for marker-assisted introgression of genomic regions associated with a purple phenotype.

Broccoli plants comprising one or more introgressed regions associated with a purple phenotype wherein at least 10%, 25%, 50%, 75%, 90%, or 99% of the remaining genomic sequences carry markers characteristic of the second genetic background are also provided. Broccoli plants comprising an introgressed region comprising regions closely linked to or adjacent to the genomic regions and markers provided herein and associated with a purple phenotype are also provided.

### III. Development of New Broccoli Varieties Having a Desirable Trait

One aspect of the present disclosure concerns methods for producing a plant with a purple phenotype. Such methods can be used for propagation of a plant comprising a recombinant chromosomal segment on chromosome 6, wherein said recombinant chromosomal segment comprises an allele that confers to said plant a purple phenotype or can be used to produce plants that are derived from such a plant. Plants derived from a plant with a purple phenotype may be used, in certain embodiments, for the development of new varieties of broccoli.

For most breeding objectives, commercial breeders work within germplasm that is of a “cultivated variety” or “elite.” This germplasm is easier to breed because it generally performs well when evaluated for horticultural performance. Numerous Brassica oleracea cultivated varieties (cultivars) have been developed, including, but not limited to, broccoli, cauliflower, sprouting broccoli, Brussels sprouts, white cabbage, red cabbage, savoy cabbage, curly kale cabbage, turnip cabbage and Portuguese cabbage. However, the performance advantage a cultivated or elite germplasm provides can be offset by a lack of allelic diversity. Breeders generally accept this tradeoff because progress is faster when working with cultivated material than when breeding with genetically diverse sources.

The process of introgressing desirable genes from non-cultivated lines into elite cultivated lines while avoiding problems with linkage drag or low heritability is a long and often arduous process. Success in deploying alleles derived from wild relatives therefore strongly depends on minimal or truncated introgressions that lack detrimental effects and reliable marker assays that replace phenotypic screens. Success is further defined by simplifying genetics for key attributes to allow focus on genetic gain for quantitative traits. Moreover, the process of introgressing genomic regions from non-cultivated lines can be greatly facilitated by the availability of informative markers.

One of skill in the art would therefore understand that the alleles, polymorphisms, and markers provided by the present disclosure allow the tracking and introduction of any of the genomic regions identified herein into any genetic background to which a broccoli species can be crossed. In addition, the genomic regions associated with the purple phenotype described herein can be introgressed from one genotype to another and tracked phenotypically or genetically. The development of the markers described herein may be used for selection of the purple phenotype and facilitates the development of broccoli plants having beneficial phenotypes. For example, plants and seeds can be genotyped using the markers of the present disclosure in order to develop varieties that exhibit a desired purple phenotype. Moreover, marker-assisted selection (MAS) allows identification of plants which are homozygous or heterozygous for the desired introgression.

Meiotic recombination is essential for plant breeding because it enables the transfer of favorable alleles across genetic backgrounds, the removal of deleterious genomic fragments, and pyramiding traits that are genetically tightly linked. In the absence of accurate markers, limited recombination forces breeders to enlarge segregating populations for progeny screens. Moreover, phenotypic evaluation is time-consuming, resource-intensive, and not reproducible in every environment, particularly for traits like plant color phenotypes. The markers provided by the present disclosure offer an effective alternative and therefore represent a significant advance in the art.

Many desirable traits that are successfully introduced through introgression can also be introduced directly into a plant by the use of molecular techniques. One aspect of the present disclosure includes plants with a genome that has been changed by any method using site-specific genome modification techniques. Techniques of site-specific genome modification include the use of enzymes such as, endonucleases, recombinases, transposases, helicases, and any combination thereof. In one aspect, an endonuclease is selected from a meganuclease, a zinc-finger nuclease (ZFN), a transcription activator-like effector nucleases (TALEN), an Argonaute, and an RNA-guided nuclease, such as a CRISPR associated nuclease.

In another aspect, the endonuclease is a dCas9-recombinase fusion protein. As used herein, a “dCas9” refers to a Cas9 endonuclease protein with one or more amino acid mutations that result in a Cas9 protein without endonuclease activity, but retaining RNA-guided site-specific DNA binding. As used herein, a “dCas9-recombinase fusion protein” is a dCas9 with a protein fused to the dCas9 in such a manner that the recombinase is catalytically active on the DNA.

Non-limiting examples of recombinase include a tyrosine recombinase attached to a DNA recognition motif provided herein is selected from the group consisting of a Cre recombinase, a Gin recombinase a Flp recombinase, and a Tnp1 recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Site-specific genome modification enzymes induce a genome modification, such as a double-stranded DNA break (DSB) or single-strand DNA break, at the target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of exogenous sequences by homologous recombination.

Another aspect of the present disclosure includes transgenic plant cells, transgenic plant tissues, transgenic plants, and transgenic seeds that comprise the recombinant DNA molecules provided by the present disclosure. Plants comprising the recombinant DNA molecules and engineered proteins, or plants produced from the cells, tissues or seeds, have curds or heads that exhibit increased resistance to downy mildew. Suitable methods for transformation of host plant cells for use with the current disclosure include virtually any method by which DNA can be introduced into a cell (for example, where a recombinant DNA construct is stably integrated into a plant chromosome) and are well known in the art. An exemplary and widely utilized method for introducing a recombinant DNA construct into plants is the Agrobacterium transformation system, which is well known to those of skill in the art. Another exemplary method for introducing a recombinant DNA construct into plants is insertion of a recombinant DNA construct into a plant genome at a pre-determined site by methods of site-directed integration. Transgenic plants can be regenerated from a transformed plant cell by the methods of plant cell culture. A transgenic plant homozygous with respect to a transgene (that is, two allelic copies of the transgene) can be obtained by self-pollinating (selfing) a transgenic plant that contains a single transgene allele with itself, for example an R0 plant, to produce RI seed. One fourth of the RI seed produced will be homozygous with respect to the transgene. Plants grown from germinating RI seed can be tested for zygosity, using a SNP assay, DNA sequencing, or a thermal amplification assay that allows for the distinction between heterozygotes and homozygotes, referred to as a zygosity assay.

New varieties may also be developed by way of double haploids. This technique allows the creation of true breeding lines without the need for multiple generations of selfing and selection. In this manner, true breeding lines can be produced in as little as one generation. Haploid embryos may be produced from microspores, pollen, anther cultures, or ovary cultures. The haploid embryos may then be doubled autonomously, or by chemical treatments (e.g. colchicine treatment). Alternatively, haploid embryos may be grown into haploid plants and treated to induce chromosome doubling. In either case, fertile homozygous plants are obtained. In accordance with the present disclosure, any of such techniques may be used in connection with a plant described herein and progeny thereof to achieve a homozygous line.

### IV. Molecular Assisted Breeding Techniques

Genetic markers that can be used in the practice of the present disclosure include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Vegetable breeders use molecular markers to interrogate a crop's genome and classify material based on genetic, rather than phenotypic, differences. Advanced marker technologies are based on genome sequences, the nucleotide order of distinct, polymorphic genotypes within a species. Such platforms enable selection for horticultural traits with markers linked to favorable alleles, in addition to the organization of germplasm using markers randomly distributed throughout the genome. In the past, a priori knowledge of the genome lacked for major vegetable crops that now have been sequenced. Scientists exploited sequence homology, rather than known polymorphisms, to develop marker platforms. Man-made DNA molecules are used to prime replication of genome fragments when hybridized pair-wise in the presence of a DNA polymerase enzyme. This synthesis is regulated by thermal cycling conditions that control hybridization and replication of DNA strands in the polymerase chain reaction (PCR) to amplify DNA fragments of a length dependent on the distance between each primer pair. These fragments are then detected as markers and commonly known examples include AFLP and RAPD. A third technique, RFLP does not include a DNA amplification step. Amplified fragment length polymorphism (AFLP) technology reduces the complexity of the genome. First, through digestive enzymes cleaving DNA strands in a sequence-specific manner. Fragments are then selected for their size and finally replicated using selective oligonucleotides, each homologous to a subset of genome fragments. As a result, AFLP technology consistently amplifies DNA fragments across genotypes, experiments and laboratories.

Polymorphisms comprising as little as a single nucleotide change can be assayed in a number of ways. For example, detection can be made by electrophoretic techniques including a single strand conformational polymorphism (Orita et al., Genomics 8:271-278, 1989), denaturing gradient gel electrophoresis (Myers, EP 0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific alleles (PASA; Sommer et al., Biotechniques 12:82-87, 1992), or PCR amplification of multiple specific alleles (PAMSA; Dutton and Sommer, Biotechniques 11:700-702, 1991).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments of methods of the disclosure, polymorphic nucleic acids can be used to detect, in a broccoli plant, a genotype associated with a purple phenotype, identify a broccoli plant with a genotype associated with purple phenotype, and to select a broccoli plant with a genotype associated with purple phenotype. In certain embodiments of methods of the disclosure, polymorphic nucleic acids can be used to produce a broccoli plant that comprises in its genome an introgressed locus associated with a purple phenotype. In certain embodiments of the disclosure, polymorphic nucleic acids can be used to breed progeny broccoli plants comprising a locus associated with a purple phenotype.

Genetic markers may include “dominant” or “codominant” markers. “Codominant” markers reveal the presence of two or more alleles (two per diploid individual). “Dominant” markers reveal the presence of only a single allele. Markers are preferably inherited in codominant fashion so that the presence of both alleles at a diploid locus, or multiple alleles in triploid or tetraploid loci, are readily detectable, and they are free of environmental variation, i.e., their heritability is 1. A marker genotype typically comprises two marker alleles at each locus in a diploid organism. The marker allelic composition of each locus can be either homozygous or heterozygous. Homozygosity is a condition where both alleles at a locus are characterized by the same nucleotide sequence. Heterozygosity refers to different conditions of the allele at a locus.

Nucleic acid-based analyses for determining the presence or absence of the genetic polymorphism (i.e., for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, alleles, or genomic regions that comprise or are linked to a genetic marker that is linked to or associated with purple phenotype in broccoli plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al., Cold Spring Harbor Symp. Quant. Biol. 51:263-273, 1986; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981; and 7,250,252 all of which are incorporated herein by reference in their entirety. However, the compositions and methods of the present disclosure can be used in conjunction with any polymorphism typing method to type polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944, where a sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is disclosed in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

### V. Edible Compositions

As used herein, “edible composition” refers to a composition which may be ingested by a mammal such as a food or feed product or a pharmaceutical composition. As used herein “food” and “feed products” refer to substances that can be used or prepared for use as food for an animal or human and include substances that may be used in the preparation of food or as food additives. Typical food or feed products include but are not limited to soups, juices, smoothies, spreads, yogurts, sauces, gravies, quiches, pies, prepared vegetable products, such as a vegetable bake, and blended vegetable products. Furthermore, edible compositions described herein may also be ingested as an additive or supplement. These can be formulated together with a nutritional substances, such as various vitamins and minerals, and incorporated into substantially liquid compositions, substantially solid compositions, or gelatins. Edible compositions as described herein may be, in some embodiments, in powder form.

As used herein “pharmaceutical composition” refers to a composition comprising a pharmaceutically acceptable carrier. In a specific embodiment, the term “pharmaceutically acceptable” refers to a carrier suitable for administration to an animal or human. The edible compositions of the present disclosure may for example comprise any pharmaceutically acceptable carrier selected with regard with the intended route of administration and standard pharmaceutical practice. The edible compositions of the present disclosure may for example be administered as a tablet, a capsule, an elixir, a solution, or a suspension.

The edible compositions described here may be produced using any method known in the art, non-limiting examples of which include mixing, blending, freezing, heating, cooling, lyophilizing, pasteurizing, and cooking using any suitable method.

The broccoli of the present disclosure can be used in any product incorporating broccoli including but not limited to the use of the broccoli, a part of the broccoli, an extract or derivative of the broccoli, or a cell of the broccoli. The broccoli can be used as an ingredient, component, formulation, extract, or derivative. The broccoli can be present as identifiable parts or non-identifiable parts. The broccoli can be fresh, frozen, or dehydrated. The broccoli can be used in any food, beverage, extract, supplement, nutraceutical, or the like for all purposes including but not limited to human or animal food, feed, or supplements. In particular, the broccoli can be used as a fresh or frozen intact broccoli product or intact or processed in a soup, ready to eat meal, snack bar, drink, smoothie, shake, tablet, capsule, injectable, vitamin, or other similar products.

### VI. Definitions

The following definitions are provided to better define the present disclosure and to guide those of ordinary skill in the art in the practice of the present disclosure. Unless otherwise noted, terms are to be understood according to conventional usage by those of ordinary skill in the relevant art.

As used herein, the term “plant” includes plant cells, plant protoplasts, plant cells of tissue culture from which plants can be regenerated, plant calli, plant clumps, and plant cells that are intact in plants or parts of plants such as an embryo, a meristem, a cotyledon, a leaf, an anther, a flower, a pistil, a seed, a stem, a stalk, a root, a shoot, a microspore, pollen or any portion thereof, or a non-regenerable portion of a plant part, including a cell, for example. As used in this context, a “non-regenerable” portion of a plant part is a portion that cannot be induced to form a whole plant or that cannot be induced to form a whole plant that is capable of sexual and/or asexual reproduction. In certain non-limiting embodiments, a non-regenerable portion of a plant or part thereof is a seed, leaf, flower, stem, root, or cell, or any portion thereof.

The term “broccoli plant” refers to broccoli plants of species Brassica oleracea var italica and encompasses broccoli varieties, breeding lines, inbred lines, hybrids, and the like.

A “variety” or “cultivar” is used herein in conformity with the International Union for the Protection of New Varieties of Plants (“UPOV”) convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

As used herein, the term “head” corresponds to the harvested part of the cultivated broccoli plant; it refers to a collection of florets attached to secondary stems, those secondary stems being arranged at the terminus of the primary stem of the broccoli plant. The head comprises one part of the primary stem, the portion of it that is above the severing point upon harvesting. A head may also be referred to as a “curd.”

The expression “primary stem” as used therein, means the main stem or stalk of the broccoli plant, at the extremity of which the secondary stems start.

The expression “secondary stem” as is used herein, means a stem that branches from the primary stem of the broccoli plant and that is supporting- and forming part of individual florets. A head comprises a plurality of secondary stems having florets at their top.

A “floret” refers to the flower buds cluster at the top of secondary stems and that is comprising part of that secondary stem supporting the flower bud cluster. An assembly of florets is comprised in a head. Florets provide a dense cluster of unopened and tight broccoli flowers buds. As used herein “floret stem,” is the stem supporting the flower bud cluster. It is not limited to the secondary stem, but also tertiary stems and others.

As used herein, “side shoot,” refers to the axillary stems except for a main stem, and supporting and forming part of individual florets.

As used herein, “floret,” refers to the flower bud cluster including that part of the secondary stem supporting the flower bud cluster, which collectively make up the curd or head.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

As used herein, a “phenotypic trait” refers to the appearance or other detectable characteristic of an individual, resulting from the interaction of its genome, proteome, and/or metabolome with the environment.

As used herein, a “purple phenotype” characterizing the broccoli plants of any of the embodiments and associated with any of the following terms “trait” or “locus” or “allele” or “QTL” means that said plants are characterized by having an intense and consistent purple color in, but not limited to, florets, flower beads, stem, main head, leaf petiole, and leaf midrib. Color classifications used to describe the plants are based on the color chart of the Royal Horticultural Society (RHS Color Chart).

As used herein, “RHS color chart,” refers to the Royal Horticultural Society (RHS) Color Chart Reference which is a standardized reference which allows accurate identification of any color. A color's designation on the chart describes its hue, brightness, and saturation. A color is precisely named by the RHS color chart by identifying the group name, sheet number, and letter, e.g., Violet Group 83A or Purple Group N77A.

As used herein, the term “locus” or “loci” refers to a specific place or places or a site on a chromosome where, for example, a gene or genetic marker is found. A “quantitative trait locus (QTL)” is a chromosomal location that encodes for at least a first allele that affects the expressivity of a phenotype.

As used herein, an “introgression fragment” or “introgression segment” or “introgression region” refers to a chromosome fragment (or chromosome part or region) which has been introduced into another plant of the same or related species by crossing or traditional breeding techniques, such as backcrossing, i.e., the introgressed fragment is the result of breeding methods referred to by the verb “to introgress.” It is understood that the term “introgression fragment” never includes a whole chromosome, but only a part of a chromosome.

A genetic element, a locus, an introgression fragment or a gene or allele conferring a trait (such as downy mildew resistance) is said to be “obtainable from” or can be “obtained from” or “derivable from” or can be “derived from” or “as present in” or “as found in” a plant or seed if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene, or allele. The terms are used interchangeably and the genetic element, locus, introgression fragment, gene, or allele can thus be transferred into any other genetic background lacking the trait. Not only seeds deposited and comprising the genetic element, locus, introgression fragment, gene, or allele can be used, but also progeny/descendants from such seeds which have been selected to retain the genetic element, locus, introgression fragment, gene, or allele, can be used and are encompassed herein, such as commercial varieties developed from the deposited seeds or from descendants thereof. Whether a plant comprises the same genetic element, locus, introgression fragment, gene, or allele as obtainable from the deposited seeds can be determined by the skilled person using one or more techniques known in the art, such as phenotypic assays, whole genome sequencing, molecular marker analysis, trait mapping, chromosome painting, allelism tests, and the like.

As used herein, a “marker” refers to detectable characteristic that can be used to discriminate between organisms. Examples of such characteristics include, but are not limited to, genetic markers, biochemical markers, metabolites, morphological characteristics, and agronomic characteristics.

As used herein, a “molecular marker” is a piece of DNA associated with a certain genomic or chromosomal location or single nucleotide polymorphism (SNP), which is found on the chromosome close to the gene of interest. Molecular markers can be used to identify a particular sequence of DNA, or a certain location in a genome or on a chromosome, or to identify an introgression fragment. When reference is made herein to one or more molecular markers being “detectable” by a molecular marker assay, this means of course that the plant or plant part comprises the one or more markers in its genome, as the marker would otherwise not be detectable.

As used herein, “flanking markers” or “bordering markers” are molecular markers located on the chromosome on either side of an allele or gene of interest, i.e., one marker on the right side of the allele or gene and one marker on the left side of the allele or gene.

As used herein, “closely linked marker” is a marker which is physically close enough to an allele or gene to co-segregate with the allele or gene at a high frequency, i.e., the chance of recombination taking place between the allele or gene and the marker is so small that the marker can be used to reliably select for the presence of the allele or gene in a breeding program (marker-assisted selection).

As used herein, “marker-assisted selection” or “MAS” refers to a process of using the presence of molecular markers, which are genetically and physically linked to a particular locus or to a particular chromosomal region (e.g., introgression fragment), to select plants (e.g., progeny) for the presence of the specific locus or region (e.g., introgression fragment).

As used herein, “marker assay” or “genotyping assay” refers to an assay which can be used to determine the marker genotype, e.g., the SNP genotype. For example, SNP markers can be detected using a KASP-assay or other assays known to the skilled person.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety means any plant or variety that has resulted from breeding for superior agronomic performance. An “elite plant” refers to a plant belonging to an elite variety. Numerous elite varieties are available and known to those of skill in the art of Brassica oleracea breeding. An “elite population” is an assortment of elite individuals or lines that can be used to represent the state of the art in terms of agronomically superior genotypes of a given crop species, such as a Brassica oleracea line. Similarly, an “elite germplasm” or elite strain of germplasm is an agronomically superior germplasm.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, the term “physical distance” referring to a region between loci (e.g., between molecular markers and/or between phenotypic markers) on the same chromosome is the actual physical distance expressed in base pairs (bp), kilobase pairs (kb), or megabase pairs (Mb).

As used herein, the term “genetic distance” between loci (e.g., between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

As used herein, the term “introgressed,” when used in reference to a genetic locus, refers to a genetic locus that has been introduced into a new genetic background, such as through backcrossing. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

“Sequence identity” and “sequence similarity” can be determined by alignment of two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters) share at least a certain minimal percentage of sequence identity. These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL (Henikoff and Henikoff, PNAS 89:10915-10919; 1992). Sequence alignments and scores for percentage sequence identity may for example be determined using computer programs, such as EMBOSS as available on the world wide web at ebi.ac.uk/Tools/psa/emboss_needle. Alternatively, sequence similarity or identity may be determined by searching against databases such as FASTA, BLAST, etc., but hits should be retrieved and aligned pairwise to compare sequence identity. Two nucleic acid sequences have “substantial sequence identity” if the percentage sequence identity is at least 85%, 90%, 95%, 98%, 99% or more (e.g. at least 99.1, 99.2, 99.3, 99.4, 99.5, 99.6, 99.7, 99.8, 99.9 (as determined by Emboss “needle” using default parameters, i.e. gap creation penalty=10, gap extension penalty=0.5, using scoring matrix DNAFULL for nucleic acids)). Markers may sometimes exhibit variation, particularly in regions which are not recognized by the probes.

As used herein, the term “denoting” when used in reference to a plant genotype refers to any method whereby a plant is indicated to have a certain genotype. This includes any means of identification of a plant having a certain genotype. Indication of a certain genotype may include, but is not limited to, any entry into any type of written or electronic medium or database whereby the plant's genotype is provided. Indications of a certain genotype may also include, but are not limited to, any method where a plant is physically marked or tagged. Illustrative examples of physical marking or tags useful in the disclosure include, but are not limited to, a barcode, a radio-frequency identification (RFID), a label, or the like.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted trait.

### VII. Deposit Information

A deposit was made of at least 625 seeds of broccoli (Brassica oleracea) line BRM-8V21-4060SI, which comprises the purple phenotype QTL on chromosome 6, as described herein. The deposit was made with the with Provasoli-Guillard National Center for Marine Algae and Microbiota (NCMA), 60 Bigelow Drive, East Boothbay, Maine, 04544 USA. The deposit is assigned NCMA Accession No. 202211063, and the date of deposit was Nov. 28, 2022.

Upon issuance of a patent, all restrictions upon the deposit will be removed, and the deposit is intended to meet all of the requirements of 37 C.F.R. §§ 1.801-1.809. The deposit has been accepted under the Budapest Treaty and will be maintained in the depositary for a period of 30 years, 5 years after the last request, or the effective life of the patent, whichever is longer, and will be replaced if necessary during that period. Applicants do not waive any infringement of their rights granted under this patent or any other form of variety protection, including the Plant Variety Protection Act (7 U.S.C. 2321 et seq.).

### Example 1

### Identification of Novel Loci Conferring the Purple Phenotype in Broccoli

A panel comprised of 223 broccoli lines and 80 cabbage lines was used for a genome wide association study (GWAS). The GWAS was performed to map genomic regions associated with the purple phenotype. In the broccoli population, two of the 223 lines included the purple phenotype and in the cabbage population, six of the 80 lines included the purple phenotype. The purple phenotype is exhibited in broccoli plant tissues including, but not limited to, the florets, main head, stems, flower bead, leaf petiole, and leaf midrib. A significant QTL was identified between the genomic positions of 18,177,971 bp to 41,865,934 bp on chromosome 6. Although this region spans a relatively large physical distance on chromosome 6, it is highly conserved across Brassica oleracea species and the genetic distance between markers M1 and M4 is small. SNP markers were identified within the region that were found to be tightly linked to the purple phenotype markers. The four markers which can be used to identify, track, or introgress the unique locus is set forth in Table 1 below.

TABLE 1
SNP markers and favorable alleles for tracking the purple phenotype locus.

| | | Position of | | SNP | | | |
|---|---|---|---|---|---|---|---|
| | | SNP in Braol | Marker | Position | | | |
| Marker | | HDEM V1.0 | Size | in Marker | SNP | Favorable | SEQ ID |
| Name | Chr. | genome (bp) | (bp) | (bp) | Change | Allele | NO. |
| M1 | 6 | 18,177,971 | 121 | 61 | C/T | C | 1 |
| M2 | 6 | 29,582,267 | 121 | 61 | C/T | C | 2 |
| M3 | 6 | 39,222,337 | 121 | 61 | T/G | T | 3 |
| M4 | 6 | 41,865,934 | 121 | 61 | G/T | G | 4 |

The boundary markers for the QTL are markers M1, a [C/T] SNP change at position 18,177,971 bp of the public Brassica oleracea genome, and M4, a [G/T] SNP change at position 41,865,934 bp. Interstitial markers M2, a [C/T] SNP change at position 29,582,267 bp, and M3, a [T/G] SNP change at position 39,222,337 bp, can be used to identify, track, or introgress the locus on chromosome 6 that confers the purple phenotype. Due to the small genetic distance between markers M1 and M4, detection of only one of the four markers described in Table 1 is sufficient to track the trait.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit, and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. An elite broccoli plant comprising a recombinant chromosomal segment on chromosome 6, wherein said recombinant chromosomal segment comprises an allele from Brassica oleracea var. capitata f. rubra that confers to said plant a purple phenotype compared to a plant not comprising said allele.

2. The plant of claim 1, wherein said recombinant chromosomal segment comprises a marker selected from the group consisting of marker M1 (SEQ ID NO:1), marker M2 (SEQ ID NO:2), marker M3 (SEQ ID NO:3), and marker M4 (SEQ ID NO:4) on chromosome 6.

3. The plant of claim 1, wherein said plant is homozygous for said recombinant chromosomal segment.

4. The plant of claim 1, wherein said plant is heterozygous for said recombinant chromosomal segment.

5. A plant part of the plant of claim 1, wherein said plant part comprises said recombinant chromosomal segment.

6. The plant part of claim 5, wherein said plant part is a cell, a seed, a root, a stem, a stalk, a leaf, a bud, a flower, a floret, a head, an ovule, or pollen.

7. A seed that produces the plant of claim 1.

8. The plant of claim 1, wherein a sample of seed comprising said recombinant chromosomal segment has been deposited under NCMA Accession No. 202211063.

9. A recombinant DNA segment comprising an allele from Brassica oleracea var. capitata f. rubra, wherein said allele confers to a broccoli plant a purple phenotype when present in said plant.

10. The recombinant DNA segment of claim 9, wherein said recombinant DNA segment comprises a sequence selected from the group consisting of SEQ ID NOs: 1, 2, 3, and 4.

11. The recombinant DNA segment of claim 9, further defined as comprised within a plant, plant part, plant cell, or seed.

12. The recombinant DNA segment of claim 9, wherein a sample of seed comprising said recombinant DNA segment has been deposited under NCMA Accession No. 202211063.

13. A method of producing an elite broccoli plant exhibiting a purple phenotype, comprising introgressing into said plant an allele from Brassica oleracea var. capitata f. rubra within a recombinant chromosomal segment flanked in the genome of said plant by marker M1 (SEQ ID NO: 1) and marker M4 (SEQ ID NO:4) on chromosome 6, wherein said allele confers to said plant a purple phenotype compared to a plant not comprising said allele, and wherein said introgressing comprises marker-assisted selection.

14. The method of claim 13, wherein said introgressing comprises:
a) crossing a plant comprising said chromosomal segment with itself or with a broccoli plant of a different genotype to produce one or more progeny plants; and
b) selecting a progeny plant comprising said chromosomal segment.

15. The method of claim 14, wherein selecting a progeny plant comprises detecting nucleic acids comprising marker locus M1 (SEQ ID NO:1), marker locus M2 (SEQ ID NO:2), marker locus M3 (SEQ ID NO:3), or marker locus M4 (SEQ ID NO:4).

16. The method of claim 14, wherein the progeny plant is an F2-F6 progeny plant.

17. The method of claim 14, wherein said crossing comprises backcrossing.

18. A broccoli plant obtainable by the method of claim 13.

19. A method of producing food or feed comprising obtaining a plant according to claim 1 or 18, or a part thereof, and producing said food or feed from said plant or part thereof.

20. A method of selecting a broccoli plant exhibiting a purple phenotype, comprising:
a) crossing the broccoli plant of claim 1 with itself or with a second broccoli plant of a different genotype to produce one or more progeny plants; and
b) selecting a progeny plant comprising said recombinant chromosomal segment.

21. The method of claim 20, wherein selecting said progeny plant comprises detecting a marker locus genetically linked to said recombinant chromosomal segment.

22. The method of claim 21, wherein selecting said progeny plant comprises detecting a marker locus within or genetically linked to a chromosomal segment flanked in the genome of said plant by marker locus M1 (SEQ ID NO:1) and marker locus M4 (SEQ ID NO:4) on chromosome 6.

23. The method of claim 20, wherein selecting a progeny plant comprises detecting nucleic acids comprising marker locus M1 (SEQ ID NO:1), marker locus M2 (SEQ ID NO:2), marker locus M3 (SEQ ID NO:3), or marker locus M4 (SEQ ID NO:4).

24. The method of claim 20, wherein said progeny plant is an F2-F6 progeny plant.

25. The method of claim 20, wherein producing said progeny plant comprises backcrossing.

26. A broccoli plant obtainable by the method of claim 20.

27. A cell according to claim 6.

28. A tissue culture comprising the cell of claim 6.

29. A method of selecting a broccoli plant or broccoli seed, said method comprising:
a) detecting in a population of broccoli plants or broccoli seeds a broccoli plant or broccoli seed comprising an allele from Brassica oleracea var. capitata f. rubra that confers to said plant a purple phenotype compared to a plant not comprising said allele.
b) selecting said broccoli plant or broccoli seed comprising said allele from Brassica oleracea var. capitata f. rubra that confers to said plant a purple phenotype.

30. The method of claim 29, wherein said allele is located on chromosome 6.

31. The method of claim 29, wherein said allele is linked to any marker selected from the group consisting of marker M1 (SEQ ID NO:1), marker M2 (SEQ ID NO:2), marker M3 (SEQ ID NO:3), and marker M4 (SEQ ID NO:4) on chromosome 6.

32. The method of any one of claims 29 to 31, wherein said plant is homozygous for said allele from Brassica oleracea var. capitata f. rubra.

33. The method of any one of claims 29 to 31, wherein said plant is heterozygous for said allele from Brassica oleracea var. capitata f. rubra.

34. The method of any one of claims 31 to 33, wherein the marker
M1 comprises a C at position 61 of SEQ ID NO:1;
M2 comprises a C at position 61 of SEQ ID NO:2;
M3 comprises a T at position 61 of SEQ ID NO:3; and
M4 comprises a G at position 61 of SEQ ID NO:4.

35. The method of any one of claims 29 to 33, wherein said broccoli plant or broccoli seed is an elite plant or a seed of an elite plant.

36. A plant identifiable by the method according to any one of claims 29 to 35.

37. A nucleic acid molecule comprising an allele conferring to a broccoli plant a purple phenotype, wherein the nucleic acid molecule is obtainable or can be obtained from seed deposited under NCMA Accession No. 202211063, and wherein the nucleic acid molecule comprises at least one marker selected from the group consisting of marker M1 (SEQ ID NO:1), marker M2 (SEQ ID NO: 2), marker M3 (SEQ ID NO:3), and marker M4 (SEQ ID NO:4).

38. The nucleic acid molecule according to claim 37, wherein the at least one marker comprises:
SEQ ID NO:1 comprising a C at position 61;
SEQ ID NO:2 comprising a C at position 61;
SEQ ID NO:3 comprising a T at position 61; or
SEQ ID NO:4 comprising a G at position 61.


SEQUENCE LISTING (4 sequences)

SEQ ID NO: 1 - DNA, 121 bp, Brassica oleracea
  source 1..121: mol_type = genomic DNA; note = Brassica oleracea var. italica; variety = italica
ccaaggagcgcattactacgccatcgactccacatccaaaccaggaagaacctcaggaaayatttggacccgaacatgtctttcgaaaagaacgccttctgcgagtttcatcaaactaaaa

SEQ ID NO: 2 - DNA, 121 bp, Brassica oleracea
  source 1..121: mol_type = genomic DNA; note = Brassica oleracea var. italica; variety = italica
aattgtagcagaactggttagttatctcgattaagatccacgaggtagttagtgacctgayttggtgtgcaacgagaacattacactttgtgaacaaaatgattttaatatctcggtttct

SEQ ID NO: 3 - DNA, 121 bp, Brassica oleracea
  source 1..121: mol_type = genomic DNA; note = Brassica oleracea var. italica; variety = italica
aagatttgcgctgagagtggggtttgtgttcaccactctctgtattagatatatctctttktttgtataggaggtcacataaagtatctggcttaattgtgtgacaggttaccccaaagaa

SEQ ID NO: 4 - DNA, 121 bp, Brassica oleracea
  source 1..121: mol_type = genomic DNA; note = Brassica oleracea var. italica; variety = italica
ttcaaatgattgtaagtaggcgactatttgtcaactcaacaggctgatttttttctttctkccggtcgtaactgggtctcgttggtttcgttgaatgggcctatttcgacttgcaaaagcc